(19) United States
(12) Patent Application Publication
(10) Pub. No.: US 2007/0248630 A1
(43) Pub. Date: Oct. 25, 2007

(54) PHARMACEUTICAL FORMULATIONS FOR IONTOPHORETIC METHOTREXATE DELIVERY

(76) Inventor: Phillip M. Friden, Bedford, MA (US)

Correspondence Address: ELMORE PATENT LAW GROUP, PC, 209 MAIN STREET, N. CHELMSFORD, MA, 01863, US

(21) Appl. No.: 11/737,568

(22) Filed: Apr. 19, 2007

Related U.S. Application Data
(60) Provisional application No. 60/793,679, filed on Apr. 20, 2006.

(51) Int. Cl. A61K 31/525; A61K 9/14

(52) U.S. Cl. 424/400; 424/487; 424/488; 514/251

(57) ABSTRACT

Pharmaceutical formulations suitable for iontophoresis that provide enhanced iontophoretic delivery of methotrexate to at least one target tissue are described and methods for administering methotrexate via iontophoresis.

### RELATED APPLICATION

This application claims the benefit of U.S. Provisional Application No. 60/793,679, filed on Apr. 20, 2006. The entire teachings of the above application are incorporated herein by reference.

### BACKGROUND OF THE INVENTION

An iontophoretic delivery system is, for example, a drug delivery system that releases drug at a controlled rate to the target tissue upon application. The advantages of systems wherein drug is delivered locally via iontophoresis are the ease of use, being relatively safe, and affording the interruption of the medication by simply peeling off or removing from the skin whenever an overdosing is suspected. The total skin surface area of adult is about 2 m². In recent years iontophoretic delivery of drugs has attracted wide attention as a better way of administering drugs for local as well as systemic effects. The design of iontophoretic delivery systems can usually be such that the side effects generally seen with the administration of conventional dosage forms are minimized.

Iontophoresis has been employed for many years as a means for applying medication locally through a patient's skin and for delivering medicaments to the eyes and ears. The application of an electric field to the skin is known to greatly enhance the ability of the drugs to penetrate the target tissue. The use of iontophoretic transdermal delivery techniques has obviated the need for hypodermic injection for some medicaments, thereby eliminating the concomitant problems of trauma, pain and risk of infection to the patient.

Iontophoresis involves the application of an electromotive force to drive or repel ions through the dermal layers into a target tissue. Particularly suitable target tissues include those adjacent to the delivery site for localized treatment. Uncharged molecules can also be delivered using iontophoresis via a process called electroosmosis.

Regardless of the charge of the medicament to be administered, an iontophoretic delivery device employs two electrodes (an anode and a cathode) in conjunction with the patient's skin to form a closed circuit between one of the electrodes (referred to herein alternatively as a “working” or “application” or “applicator” electrode) which is positioned at the site of drug delivery and a passive or “grounding” electrode affixed to a second site on the skin to enhance the rate of penetration of the medicament into the skin adjacent to the applicator electrode.

U.S. Pat. No. 6,477,410 to Henley et al. describe the use of iontophoresis for drug delivery. However, improved formulations that facilitate the delivery of specific active agents is desired.

### SUMMARY OF THE INVENTION

The present invention provides pharmaceutical formulations suitable for iontophoresis that provide enhanced iontophoretic delivery of methotrexate (MTX) to at least one target tissue. The formulations are further characterized by good to excellent stability. The present invention also provides methods of administering methotrexate in at least one target tissue of and/or treating psoriasis in a patient by iontophoretically delivering a formulation of the invention.

### BRIEF DESCRIPTION OF THE DRAWINGS

FIG. 1 illustrates the cumulative amount of MTX delivered across hairless rat skin increased with decrease in ionic strength of the buffer.

FIG. 2 shows that the amount of drug delivered increased with increase in MTX drug concentration from 10 to 15 mg/ml at 0.25 M buffer strength.

FIG. 3 plots the experimental data of the fractional factorial design.

FIG. 4 illustrates the response surface plot.

### DETAILED DESCRIPTION OF THE INVENTION

In one aspect, the invention provides pharmaceutical formulations that are suitable for iontophoresis and that provide enhanced iontophoretic delivery of MTX to a patient, preferably a human patient, in need of treatment.

Methotrexate (MTX) is a folic acid antagonist with antineoplastic activity that is useful in the treatment of psoriasis and rheumatoid arthritis. It is generally administered by oral or parenteral route for the treatment of psoriasis. When administered systemically over prolonged periods, it may produce side effects like nausea, vomiting, fatigue, anemia headache, leukopenia, thrombocytopenia and hepatic toxicity. Iontophoretic delivery of MTX can reduce such side effects by delivering it directly to the diseased psoriatic skin rather than systemically. Methotrexate has three pKa's (5.6, 3.8, and 4.8); it is negatively charged at physiological pH and was thus delivered by cathodal iontophoresis by means of electrorepulsion. MTX delivery was first studied at various buffer strengths and at different drug concentrations. Optimum ionic strength and drug concentration were selected and the effect of current density and time of application was studied.

Thus, the invention relates to the iontophoretic delivery of MTX. In a preferred embodiment, the MTX is formulated at a pH between 7 and 8 (e.g., 7.4). While it is preferred to not use buffers due to the possible competing ion effect, buffers can be used. Phosphate buffer is preferred. Preferably the ionic strength of the buffer is at least about 0.1M, such as about 0.25M. Preferably, the concentration of MTX in the formulation is at least about 1 mg/ml, such as at least about 8 mg/ml, preferably at least about 10 to 25 mg/ml, such as between 10 to 15 or 20 mg/ml.

A formulation of the invention is preferably a viscous formulation. As used herein, the term “viscous formulation” includes colloidal and gel formulations, such as a viscous formulation having a viscosity of greater than about 500 cp at 25 degrees Celsius. A viscosity modifying agent can be added to the formulation to achieve the desired viscosity. The pharmaceutically acceptable carrier or excipient may comprise about 0.1 to 10 weight percent of a viscosity modulating agent. As used herein, the term “pharmaceutically acceptable carrier or excipient” means any non-toxic diluent or other formulation auxiliary that is suitable for use in iontophoresis. Examples of pharmaceutically acceptable carriers or excipients include but are not limited to: diluents such as water, or other solvents, cosolvents; solubilizing agents such as sorbital and glycerin; buffers such as, for example, phosphate buffer solutions; pharmaceutically acceptable bases; and viscosity modulating agents such as cellulose and its derivatives.

As used herein the term “target tissue” includes the patient's dermis, epidermis, nails, mucocutaneous membranes including, but not limited to, the eye and the body cavity and canal sites such as mouth, ear, nose, vagina, and rectum.

The viscosity of the viscous formulation may be controlled by a viscosity modulating agent. A viscosity modulating agent includes any agent that is capable of modulating the viscosity of a gel. Viscosity modulating agents useful in the practice of the invention include but are not limited to, ionic and non-ionic, high viscosity, water soluble polymers; crosslinked acrylic acid polymers such as the “carbomer” family of polymers, e.g., carboxypolyalkylenes that may be obtained commercially under the Carbopol® trademark; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; gums such as tragacanth and xanthan gum; sodium alginate; gelatin, hyaluronic acid and salts thereof, chitosans, gellans or any combination thereof. It is preferred that non-acidic viscosity enhancing agents, such as a neutral or basic agent be employed in order to facilitate achieving the desired pH of the formulation. If a uniform gel is desired, dispersing agents such as alcohol, sorbitol or glycerin can be added, or the gelling agent can be dispersed by trituration, mechanical mixing, or stirring, or combinations thereof. In one embodiment, the viscosity enhancing agent can also provide the base, discussed above.

In one preferred embodiment, the viscosity modulating agent is cellulose that has been modified such as by etherification or esterification. One such etherified cellulose polymer is sold under the trademark Natrosol® (Hercules-Aqualon, Wilmington, Del.).

Preferred iontophoretic delivery devices useful with the compositions and methods of the invention include but are not limited to those described in U.S. Pat. Nos. 6,148,231, 6,385,487, 6,477,410, 6,553,253, and U.S. Patent Publication Numbers 2004/0111051, 2003/0199808, 2004/0039328, 2002/0161324, and U.S. Application Ser. No. 60/743,528, all incorporated herein by reference. A preferred applicator which has been developed for use with a device for electrokinetically delivering a medicament to a treatment site comprising an applicator head having opposite faces and including an active electrode and a porous pad (such as a woven or non-woven polymer, for example, polypropylene, pad); a margin of the applicator head about the active electrode having a plurality of spaced projections therealong; the porous pad and the applicator head being ultrasonically welded to one another about the margin of the head with the electrode underlying the porous pad; and a medicament or a medicament and an electrically conductive carrier therefor carried by the porous pad in electrical contact with the electrode. Alternatively or additionally, the applicator has been developed for use with a device for electrokinetically delivering a medicament to a treatment site comprising an applicator head having opposite faces and including an active electrode and a porous pad overlying the active electrode; a medicament or a medicament and an electrically conductive carrier therefor carried by the pad and in electrical contact with the electrode; a lid overlying the porous pad on a side of the porous pad remote from the electrode and releasably secured to the applicator head; and the lid comprising layers of different materials and including one or more tabs, one of the layers of the lid and the tab being formed of a metallic material, at least a portion of an interface between the metallic material of the tab and the metallic material of the lid having a discontinuity. In another embodiment, the lid may be an oversided disc having a rim constituting an annular tab. Additionally or alternatively, the applicator which has been developed for use with a device for electrokinetically delivering a medicament to a treatment site comprising an applicator head having opposite first and second faces and including an active electrode and a porous pad overlying said electrode; a medicament or a medicament and an electrically conductive carrier therefor carried by the pad; a margin of the cartridge about the active electrode and a margin of the porous pad being secured to one another; the active electrode having a first portion thereof exposed through the first face of the applicator head remote from the porous pad; and another portion of the active electrode being exposed to the porous pad along the second face of the applicator head for electrical contact with the medicament or the medicament and the electrically conductive carrier.

The formulations are preferably administered via iontophoresis. In a preferred embodiment, a current density of at least 0.02 mA/cm² is applied, such as at least 0.5 mA/cm². In a preferred embodiment, a flux of at least about 0.2 μg/cm²-hr, such as about 0.6 μg/cm²-hr, is achieved. The iontophoresis can be applied for a sufficient time to achieve an effective amount. In general, the time of application can be between about 5 and 60 minutes, such as about 30 minutes or less.

The following Examples further illustrate the present invention but should not be construed as in any way limiting its scope.

### EXAMPLES

### Example 1

### In Vitro Iontophoretic Delivery of MTX Through Nude Rat Skin

The delivery of MTX under 30 mins of iontophoresis (0.4 mA/cm²) was studied at buffer strengths 0.05-0.5 M and drug concentrations 10-20 mg/ml. The effect of current density and time of application on the delivery of MTX is studied by optimizing the full-factorial design. The current density (X₁) and time of application (X₂) are the independent factors chosen for the factorial design with 4 and 5 levels, respectively in the range of 0.05-0.5 mA (X₁) and 10-120 mins (X₂).

In vitro iontophoretic experiments (n≧3) were performed using freshly excised hairless rat skin mounted on vertical Franz diffusion cells with stratum corneum facing towards the donor chamber. The donor compartment contained 15 mg/ml of MTX in phosphate buffer of pH 7.4 (0.25 M) and the receptor compartment contained phosphate buffer of pH 7.4 (0.25 M) with 75 mM NaCl.

A silver-silver chloride electrode (cathode) was placed in the donor chamber and silver wire (anode) was placed in the receptor compartment. Samples were taken at periodic intervals and were analyzed by HPLC, using YMC RP 18 column. Mobile Phase was composed of monobasic sodium phosphate (10%) and Tris HCl buffer (90%). Flow rate was 1.5 ml/min and detection wavelength was 303 nm.

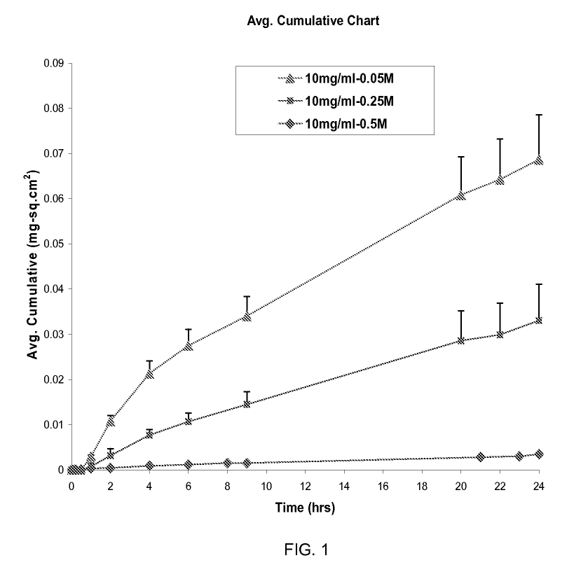

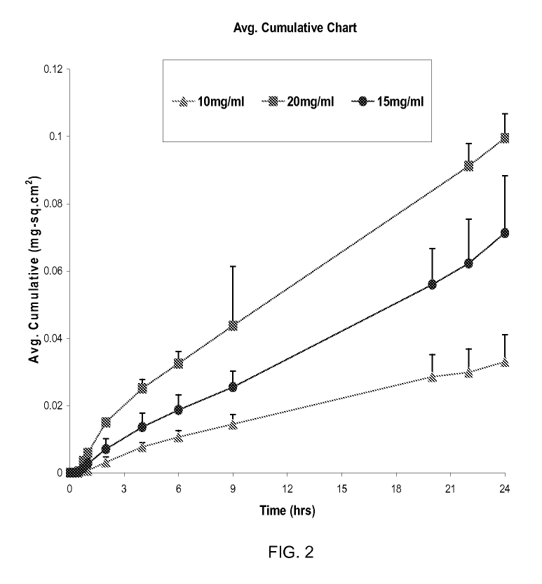

The cumulative amount of MTX delivered across hairless rat skin increased with decrease in ionic strength of the buffer (FIG. 1). Phosphate buffer of pH 7.4 and ionic strength 0.25 M was selected to avoid the changes in pH observed when 0.05 M buffer was used. The amount of drug delivered increased with increase in MTX drug concentration from 10 to 15 mg/ml at 0.25 M buffer strength (FIG. 2). However, delivery at 15 and 20 mg/ml is not statistically different; this might be due to the saturation of the boundary layer at higher concentrations.

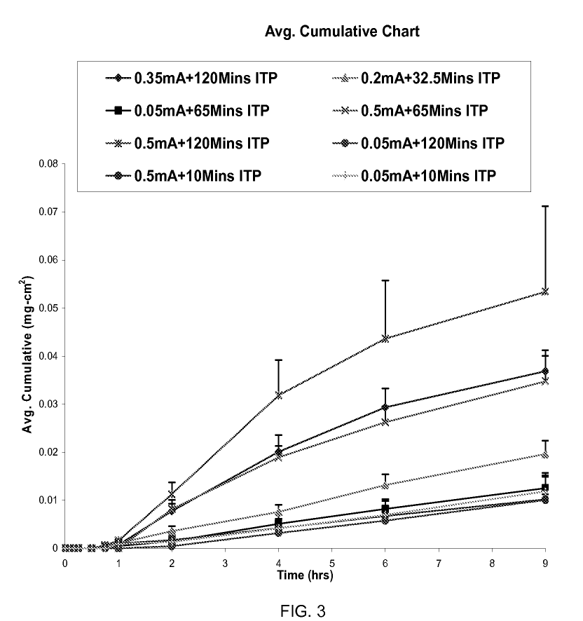

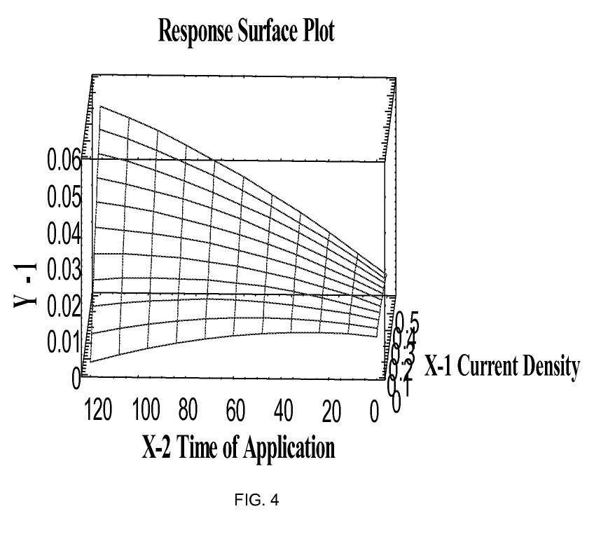

The experimental data of the fractional factorial design is plotted as seen in FIG. 3 and the response surface plot is shown in FIG. 4. The factorial design shows that with the increase in current density and time of application, the cumulative amount of MTX delivered also increased. However, for 10 min iontophoresis, an increase in current density from 0.05 to 0.5 mA did not show a statistically significant increase. An increase in time of application from 10-120 mins. at 0.05 mA did not show any enhanced delivery of the drug.

The predictions of the model have been verified by performing an experiment in the experimental region with current density 0.4 mA/cm² and ITP for 30 mins. The cumulative amount of drug delivered was found to be in between the lower and upper 95% confidence level, thus establishing the validity of the model.

Screening studies were first used to select the buffer strength (0.25 M) and drug concentration (15 mg/ml). An increase in time of current application and/or current density led to increased delivery of MTX across the skin only for higher level settings. The maximal flux of about 0.6 μg/cm²-hr was obtained.

### References

M. J. Alvarez-Figueroa, M. B. Delgado-Charro, J. Blanco-Mendez, Int. J. Pharmaceutics, 212 (2001) 101-107.
S. B. Tiwari, B. C. Kumar, N. Udupa, C. Balachandran, Int. J. Dermatology, 42 (2003) 157-159.
R. S. Upasani and A. K. Banga, Pharm. Research, 21 (2004), 2293-2299.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

As used in this specification and the appended claims, the singular forms “a”, “an”, and “the” include plural referents unless the context clearly dictates otherwise.

While this invention has been particularly shown and described with references to preferred embodiments thereof, it will be understood by those skilled in the art that various changes in form and details may be made therein without departing from the scope of the invention encompassed by the appended claims.

What is claimed:

1. A viscous formulation suitable for iontophoresis comprising methotrexate in an optional phosphate buffer at an ionic strength of at least about 0.1M.

2. The formulation of claim 1 wherein the phosphate buffer is present at an ionic strength of about 0.25M.

3. The formulation of claim 1 wherein methotrexate is present in a concentration of at least 8 mg/ml.

4. The formulation of claim 3 further comprising water.

5. The formulation of claim 4 wherein the viscosity of the formulation is no less than about 500 cp at 25 degrees Celsius.

6. The formulation of claim 5 wherein the formulation comprises a viscosity modulating agent selected from, cellulosic polymers and derivatives thereof, crosslinked acrylic acid polymers, hydrophilic polymers, gums, sodium alginate; gelatin and any combination thereof.

7. A method for administering methotrexate to a patient comprising iontophoretically administering to the body surface of a patient in need thereof, the formulation of claim 1.

8. The method of claim 7 wherein a current density of at least about 0.02 mA is applied.

9. The method of claim 8 wherein the current density is at least 0.5 mA.

10. The method of claim 7 wherein the methotrexate is administered at a flux of at least about 0.2 μg/cm²-hr.

11. The method of claim 10 wherein the flux is about 0.6 μg/cm²-hr.

12. A formulation according to claim 1 further comprising a porous pad.

13. An iontophoretic device the improvement comprising a formulation according to claim 1 absorbed onto a porous pad.

14. The method of treating psoriasis comprising iontophoretically administering to the body surface of a patient in need thereof, the formulation of claim 1.
